(19) United States
(12) Patent Application Publication
(10) Pub. No.: US 2004/0096859 A1
(43) Pub. Date: May 20, 2004

(54) Method for detecting and/or quantifying an analyte

(76) Inventors: Emil Palecek, Brno (CZ); Jorg Hassmann, Erlangen (DE)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/467,884

(22) PCT Filed: Feb. 8, 2002
(86) PCT No.: PCT/EP02/01324

(30) Foreign Application Priority Data
Feb. 12, 2001 (DE) 101 06 654.6

(51) Int. Cl. C12Q 1/68

(52) U.S. Cl. 435/6

(57) ABSTRACT

The invention relates to a method for detecting and/or quantifying a nucleic acid in a liquid. Said method comprises the following steps: a) either first microparticles and a probe having a specific affinity for the nucleic acid and for the first microparticles are prepared, or second microparticles are prepared having a probe which is bound to the surface thereof; b) a first solution containing the nucleic acid, the probe and the first microparticles is produced under conditions in which the probe binds to the nucleic acid and to the first microparticles, or a first solution containing the nucleic acid and the second microparticles is produced under conditions in which the nucleic acid binds to the probe; c) the first or second microparticles are separated from the first solution; and d) the nucleic acid is detected by means of an electrochemical method whereby the first or second microparticles are transferred into a second solution in order to detect the nucleic acid.

The invention relates to a method for detecting and/or quantifying an analyte.

A method for detecting an analyte is known in the prior art from U.S. Pat. No. 6,100,045. This entails an analyte-containing first solution being brought into contact with an analyte having a redox-active label, and with an electrode and magnetic microparticles having binding specificity for the analyte. The electrode can be produced from carbon-based ink paste. The magnetic particles are magnetically immobilized in the direct vicinity of the electrode. The binding of the analyte to the solid phase is detected by means of the redox-active label as amperometric signal via the electrode. The method can be carried out in a flow cell. It is possible for the magnetic particles and the electrode to be flushed with a substrate solution for the electrochemical detection.—The known method is not particularly sensitive. An apparatus with a relatively complicated design is necessary to carry it out.

It is furthermore known, for example from U.S. Pat. No. 5,871,918, to hybridize an analyte, e.g. a DNA, with a probe bound to an electrode. The hybridization is detected via redox indicators. WO 89/10556 describes the possibility of detecting the hybridization also via the DNA conductivity, which is increased in the hybridization state.—The hybridization of a DNA with a probe bound to the electrode surface is not always possible, because interfering third molecules attach themselves to the electrode. These methods are not particularly sensitive either.

DE 198 28 441 A1 discloses a method for detecting an analyte in a sample by electrochemiluminescence. For this purpose, the analyte is brought into contact with an analyte-specific receptor containing an electrochemiluminescent label. An osmium complex can be used as electrochemiluminescent label.

DE 44 39 345 A1 discloses conjugates which comprise luminescent osmium chelates as labeling groups for an electrochemiluminescence measurement.

DE 197 30 497 A1 discloses a magnetic pin by means of which magnetic particles with target molecules bound thereto can be extracted from a solution and transferred into another solution.

DE 198 23 719 A1 discloses a method for the processing of substances in the reservoir of a metering device, in which sample molecules are concentrated on magnetic particles in the reservoir. After the processing, the bound sample molecules can be eluted from the magnetic particles using an eluent.

DE 196 02 078 A1 discloses electrodes for amperometric competitive enzyme immunoassays which are covered with a semipermeable layer. The electrodes may be carbon paste electrodes or gold electrodes.

DE 42 16 696 C2 discloses a competitive detection method in which the analyte competes with an analyte having a redox-active label for a binding. The displaced or unbound labeled analyte can be concentrated and detected by means of cyclic voltammetry.

U.S. Pat. No. 5,770,369 discloses nucleic acids having redox-active moieties such as transition metal complexes. Electron donor and electron acceptor moieties are covalently linked at pre-defined positions. The molecules are able to transfer electrons over relatively large distances. The transition metal in the complexes may be, for example, osmium.

It is an object of the invention to eliminate the disadvantages of the prior art. It is intended in particular to indicate a method which can be carried out as quickly and simply as possible and has increased sensitivity for detecting an analyte.

This object is achieved by the features of claim 1. Expedient embodiments are evident from the features of claims 2 to 24.

The invention provides a method for detecting and/or quantifying an analyte in a liquid, in particular nucleic acids, having the following steps:

a) provision of first microparticles and of a probe which has a specific affinity for the analyte and for the first microparticles, or of second microparticles having the probe linked to the surface thereof,

b) preparation of a first solution containing the analyte, the probe and the first microparticles under conditions under which the probe binds to the analyte and to the first microparticles, or of a first solution containing the analyte and the second microparticles under conditions under which the analyte binds to the probe,

c) separation of the first or second microparticles from the first solution,

d) detection of the analyte by means of an electrochemical method, where the first or second microparticles are transferred into a second solution for detecting the analyte.

The term “analyte” includes in particular nucleic acids, hormones, antibodies, antigens of pathogenic substances, medicaments, antibiotics and the like. A probe means a molecule which binds the analyte or has a specific binding affinity for the analyte. Possible examples of the probe are antibodies, antigens, fragments of antibodies, receptors, nucleic acids or ligands.

In step b), the first microparticles, the probe and the analyte can be mixed in any sequence. It is possible, for example, first to incubate the probe with the analyte so that the probe binds to the analyte. Only then are the microparticles added, and the probe with the analyte bound thereto then binds to them.

The first and second microparticles are particles having an average diameter in the range from 0.1 to 200 μm, preferably from 1 to 20 μm, and being suitable for binding probes on their surface. Transfer into the second solution is possible by introducing the microparticles into the second solution, which is kept separate from the first solution and in which the electrochemical method is then carried out for the detection. The second solution can have its composition optimized in relation to the detection of the analyte by means of the electrochemical method. It is possible in particular for interference by unwanted foreign substances which are present where appropriate in the first solution with the electrochemical detection of the analyte to be substantially precluded. This is achieved by detection of the analyte taking place only in the second solution, so that the electrode used for the electrochemical method comes into contact only with the second solution, and not with the first. The first solution, e.g. a body fluid, may, besides the analyte, contain substances which would bind nonspecifically to the electrode on contact therewith. These substances may generate signals in the electrochemical detection which interfere with or completely obliterate the specific detection of the analyte. The nonspecific binding of some substances to the electrode may be so strong that they can scarcely be removed by rinsing or washing the electrode. A further advantage of the method is that it makes it possible to use an electrode which is not compatible, e.g. chemically, with the first solution. It is possible, because of the particularly high sensitivity of the method of the invention, to dispense with amplification of the analyte. Direct electrochemical detection of the analyte in its biologically relevant concentration is possible.

The method of the invention can be carried out quickly and simply. It is highly sensitive. The high sensitivity is made possible by the separation of the first or second microparticles from the first solution and the detection of the analyte by means of an electrochemical method.

In one embodiment, which can also be combined with the following embodiments relating to the detection of the analyte, the first or second microparticles are designed to be magnetic. In this case they are expediently the “magnetic beads” known in the art. The magnetic design of the first or second microparticles facilitates separation thereof from the first solution.

The binding of the probe to the first microparticles can take place by means of biotin, streptavidin or avidin. For this purpose, one of these molecules is linked to the probe. The first microparticles have a counter-molecule which specifically binds to these molecules. For example, biotin can be linked to the probe and avidin to the first microparticles.

In a further expedient embodiment, the analyte or the probe is labeled with complex compounds containing osmium, preferably osmium tretroxide [sic]. This makes electrochemical detection of the analyte possible in a simple manner. The complex compound can be linked, preferably terminally, to the analyte or the probe. A further possibility is to label the probe with cysteine. This, in combination with the use of mercury-containing electrodes, enables an electrochemical signal which is brought about by catalytic hydrogen evolution and which is suitable for detecting the analyte.

In a further embodiment, the binding of the analyte to the probe can be followed by addition of a reporter probe labeled with cysteine or osmium complex compounds, so that the reporter probe hybridizes with a single-stranded overhang of the analyte. The reporter probe can be removed from the analyte and subsequently detected electrochemically.

It is possible in a further embodiment for a first antibody specific for the analyte to be added to the second solution for the detection. An enzyme which chemically modifies the analyte or the first antibody, preferably peroxidase may also be added to the second solution. The chemical modification of the analyte may lead for example to an electroactive product which generates a catalytic signal. On use of DNA as analyte, the chemical modification may also consist of the DNA becoming immunogenic. The immunogenic DNA can be detected for example via enzymes, such as, for example, peroxidase coupled to specific antibodies. A second antibody specifically binding to the first antibody may also be added to the second solution. The second antibody is preferably labeled.

According to a particularly advantageous embodiment feature, the analyte in the second solution is hydrolyzed by acid. When DNA is used as analyte, the purine bases are released in the hydrolysis. The released purine bases form insoluble complexes with mercury. Complexes of this type can be detected in extremely low concentrations for example on a hanging mercury drop electrode (HMDE) or other mercury-containing electrodes by means of suitable electrochemical methods.

It has furthermore proved advantageous in step d) to apply a magnetic or electric field so that the first or second microparticles or the analyte are moved into the vicinity of an electrode. The first or second microparticles can be bound to the electrode or kept in the vicinity thereof. The aforementioned features also contribute to speeding up the method.

It is expedient to apply for a preset period an opposite magnetic or electric field so that molecules interfering with the electrochemical detection or first or second microparticles are moved away from the electrode. The application of the field and of the opposite field can take place cyclically. This increases the sensitivity of the method further.

According to a further embodiment feature, the electrode comprises at least one of the following materials: electrically conductive plastic and/or polymers, mercury, gold, carbon or indium-tin oxide. The aforementioned materials have proved to be particularly suitable for carrying out a sensitive measurement.

It is possible to provide a layer or a membrane for retaining molecules of a preset size on or in front of the surface of the electrode and/or in front of a measuring cell containing the electrode. The provision of such a layer or membrane is advantageous especially when the analyte is split up by acid treatment or by addition of an enzyme into small fragments or by acid treatment into purine bases. In this case, large molecules interfering with the electrochemical detection can be kept away from the surface by means of the layer or the membrane, while for example the purine bases pass through the layer or the membrane and reach the surface of the electrode. It is thus possible to increase the sensitivity of the method further. A membrane or layer designed with a narrow mesh can also serve to keep the analyte away from the electrode surface and thus protect it from damage by nascent gases formed on the electrode, such as, for example, oxygen and hydrogen.

The electrochemical detection method which has proved particularly advantageous is cathodic stripping voltammetry (CSV). It is possible thereby to detect, for example, purine bases in a concentration range below 10⁻⁸ M.

It is also expedient to use the electrochemical detection method to identify hydrolysis products of the analyte by means of their specific redox characteristics. Thus, for example, adenine can be detected unambiguously as constituent of a hydrolyzed analyte on the basis of its specific redox signal. In a further advantageous embodiment, the analyte can be concentrated or purified by means of a competitive assay. This measure also contributes to increasing the sensitivity of the method.

In a preferred embodiment, the analyte is amplified before or during step b) by means of a nucleic acid amplification reaction, especially a PCR. The analyte detected in step d) is then essentially the product of the amplification reaction. This also applies when the product is not, because of the primers used for the amplification reaction, completely identical to the analyte, or the product of the amplification reaction is complementary to the analyte. The probe in such a method may be a primer employed in the amplification reaction. The probe may, instead of a specific affinity for the analyte, also have a specific affinity for an opposite strand, formed in the amplification reaction, of the analyte which is complementary to the analyte. The conditions in step b) must then be chosen so that the probe binds to this opposite strand.

### GENERAL DESCRIPTION OF DIVERSE VARIANTS OF THE METHOD

A. Detection of a DNA by Detecting the Purine Bases by Means of Cathodic Stripping Voltammetry (CSV)

The purine bases adenine and guanine can be detached from a target DNA by treatment with acid. The purine bases form insoluble complexes with mercury. Such complexes can be detected even in very low concentrations below 10⁻⁸ M on a hanging mercury drop electrode (HMDE) or other mercury-containing electrodes by means of CSV, without removing the remaining DNA beforehand. If the target DNA has a distinctly greater length than the probe, it is possible and advantageous to neglect the presence of the probe.

If larger molecules interfering with the electrochemical detection of the purine bases are present in the biological material to be investigated, the electrode can be surrounded by a semipermeable membrane. Larger molecules are retained by the semipermeable membrane, while the smaller ones, such as the purine bases, are able to reach the electrode unimpeded.

B. Detection of a DNA by Chemical Modification and Subsequent Electrochemical Detection.

To carry out the method of the invention, the analyte, e.g. a DNA, is initially chemically modified. Any chemical modification which leads to an electroactive product is suitable. However, it is advantageous for the product to generate a catalytic signal or make the analyte immunogenic. The chemical modification may be designed as follows:

B.1 Modification of Bound DNA or Other Nucleic Acids by Osmium Tetroxide Complexes

It is possible to incorporate an osmium tetroxide complex (Os,L) in single-stranded DNA (ssDNA) as electroactive marker under physiological conditions. One possibility in this connection is osmium tetroxide, 2,2′-bipyridine. The incorporated Os,L causes a strong signal brought about by catalytic hydrogen evolution. This makes it possible to detect ssDNA down to a concentration of 500 pg/ml.

Os,L can be employed both as base-specific marker, specifically for thymine, and as single strand-specific marker. A further possibility is to use labeled probes which bind to DNA in a secondary process (“reporter probes”). The detection is possible not only through the DNA itself, the osmium-containing complexes or through these catalyzed processes via an immunological secondary step:

B.1.1 Base-Specific Marker

Oligonucleotides and polynucleotides are labeled at their ends with Os,L if they have a Tₙ tail and no further pyrimidine base is to be found in the remaining molecule (R) which is to undergo DNA hybridization. If, besides guanine and adenine, also some cytonsines [sic] are additionally present in R, it is necessary to use specifically adapted experimental conditions.

B.1.2 Single Strand-Specific Marker

Nucleic acids can be labeled at the end with an Os,L. In the case of a double-stranded DNA, a single-stranded Tₙ overhang or at least a T-containing overhang is necessary for the labeling. The modification must take place under conditions which do not impair the stability of the DNA (or RNA, PNA, etc.) double helix, i.e. at sufficiently high ionic strength (for DNA and RNA), a virtually neutral pH, and a temperature which is not too high (e.g. at 37° C.). If a single-stranded probe is used, the double-stranded DNA must be denatured after the modification with Os,L, and the strand modified with Os,L must be isolated.

In the two aforementioned labeling methods, the number of markers on the nucleic acid ends both in the probe molecules and in the single strand can be controlled through the amount of thymine which are [sic] attached to the molecule ends. A Tₙ overhang determines, depending on the position of the Tₙ overhang, whether the 5′ or 3′ end is labeled. The attachment of thymine residues to one end of the DNA makes the molecule electroactive and immunogenic. The thymine overhangs do not impair the hybridization of the remainder of the molecule.

B.1.3 Reporter Probes

It is also possible to employ reporter probes, i.e. further probes labeled with Os,L complexes and binding in a secondary process to the DNA to be detected, for labeling DNA to be detected.

The DNA to be detected has a single-stranded overhang after the hybridization with the probe. It is possible to hybridize thereon short-chain DNA molecules which are complementary to the overhang and which are preferably provided with Os,L complexes on the poly(dT) tail (reporter probe). After the extraction of the beads carrying the probes, the reporter probes can be removed from the target DNA in a second solution and then the Os,L complexes can be detected electrochemically.

The sensitivity can be increased additionally by an extension of the poly(dT) tail of the reporter probes. In this case it is possible to hybridize simultaneously to a plurality of relevant sequences of the target DNA; it is thus possible to obtain information for example about point defects or about a plurality of sequences.

B.1.4 Detection Via Catalytic Signals and Immunological Reactions

It is also possible for the bound target DNA to be treated with an Os,L complex such as, for example, 2,2′-bipyridine (Os,bipy) and, after a washing step, to be removed from the first or second microparticles. The electrochemical detection is possible directly with the aid of the catalytic signal of the DNA-Os,L complex, or indirectly via specific antibodies labeled with an enzyme. The antibody binds to the DNA-Os,L complex, and the complex is detected electrochemically via an enzymatic reaction. The indirect immunochemical method differs from the direct detection by requiring no HMDE, but can be measured using various other solid electrodes.

C. Detection of the Analyte by Means of Labeled Probes Generating a Catalytic Signal

The analyte, e.g. a nucleic acid, can also be labeled with cysteine. A label of this type is referred to in the case of DNA and PNA hereinafter respectively as cys-DNA and cys-PNA. Such a label results in generation of an electrochemical signal on mercury-containing electrodes through catalytic hydrogen evolution. The signal is highly specific for example for cys-PNA or cys-DNA; it is not generated by pure nucleic acids. The limits [sic] of detection for cys-PNA is at a concentration of less than 200 pg/ml. The limit of detection is to be assumed to be similar in the case of cys-DNA. If the analysis is carried out in a 5 μl drop, cys-PNA can thus be detected even at a concentration of 400 attomol. The amount of cys-PNA which interacts with the electrode surface is even distinctly smaller. Cysteine-labeled probes can be employed as reporter probes in analogy to the process described in B.1.4.

The invention is explained below by means of exemplary embodiments and the drawings. These show:

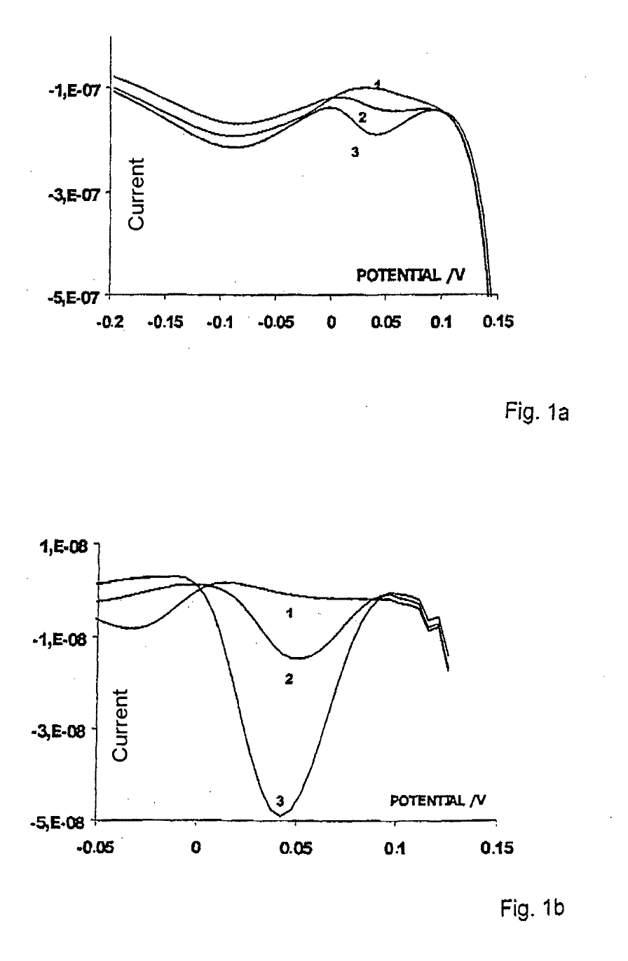

FIGS. 1a and b a first CSV voltammogram,

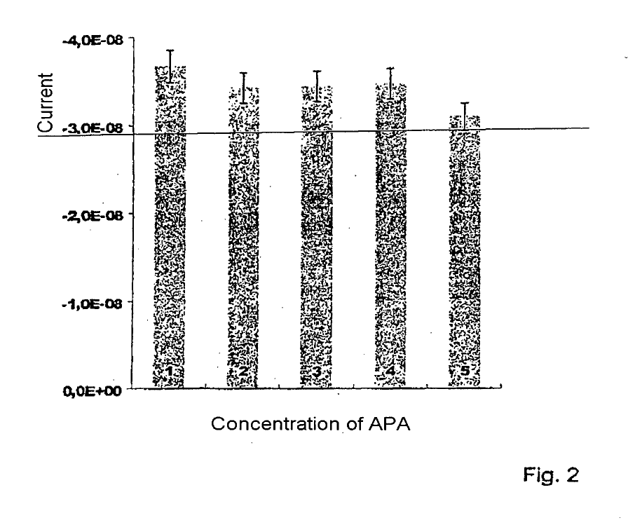

FIG. 2 the effect of the concentration of aphorinic [sic] acid (APA) on the determination of adenine,

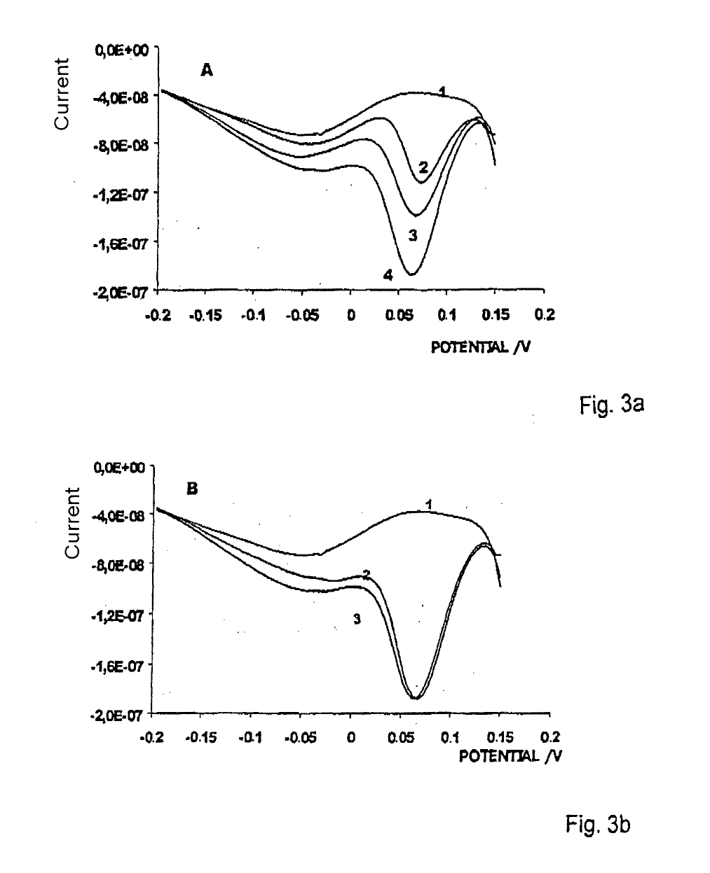

FIGS. 3a and b a second CSV voltammogram and

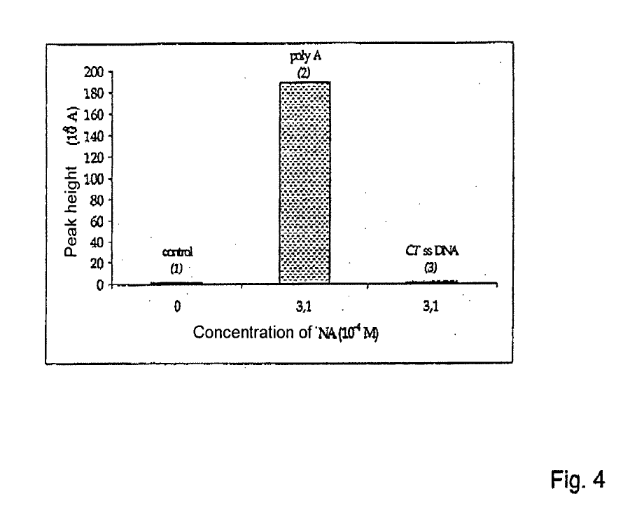

FIG. 4 a comparison of the specificity of the binding of Poly A and CT ss DNA

FIG. 1a shows a DPCS voltammogram of adenine at a concentration of 1.2 and 2.4×10⁻⁸ M without baseline correction; FIG. 1b shows the DPCS voltammogram of FIG. 1a with moving average baseline correction. Curve 1 represents in each case the electrolyte background, curve 2 corresponds to 1.2×10⁻⁸ M adenine and curve 3 is a measurement on 2.4×10⁻⁸ M adenine; Eᵢ=−0.2 V, t=5 min, v=5 mV/s, A=50 mV, 0.05 M borate buffer.

FIG. 2 shows the effect of apurinic acid (APA) on the determination of adenine. The appropriate aliquots of APA were added successively to a 2.4×10⁻⁸ M adenine solution: bar 1: 2.4×10⁻⁸ M adenine (without APA); bar 2: plus 1.2×10⁻⁸ M APA; bar 3: plus 2.4×10⁻⁸ M APA; bar 4: plus 1.2×10⁻⁷ M APA; bar 5: plus 9.1×10⁻⁷ M APA. The stated concentration of APA is based on the monomer content. DPSCV, Eᵢ=0.15 V; t_{A}=5 min, v=5 mV/s, A=50 mV.

FIG. 3a shows a comparison of equimolar concentrations of guanine and adenine with depurinated DNA. The curve [sic] represents the electrolyte background. Curve 2: 4.9×10⁻⁸ M adenine (without APA); curve 3: 4.9×10⁻⁸ M adenine plus 3.7×10⁻⁸ M guanine; curve 4: depurinated single-stranded DNA (ssDNA) (4.3×10⁻⁸ M). In FIG. 3b, curve 1 shows the electrolyte background; curve 2: depurinated ssDNA; curve 3: depurinated dsDNA (both samples 4.3×10⁻⁸ M). The DNA concentrations are based on the monomer content. DPSCV, Eᵢ=0.15 V; t_{A}=5 min, v=5 mV/s, A=50 mV.

FIG. 4 shows a comparison of CT ssDNA with poly(A) RNA. 15 μl of 3.1×10⁻⁴ M (2) poly(A) or (3) CT ssDNA were hybridized with 15 μl of DB in binding buffer. 15 μl of DB was employed as control in the binding buffer without nucleic acids.

The subsequent procedure was carried out [lacuna] exemplary embodiment 1 below. Compared with poly(A) RNA, virtually no adenine signal was obtained with CT ssDNA.

### 1st Exemplary Embodiment

The first exemplary embodiment relates to the hybridization of poly(A)-ODNs and [sic] the surface of magnetic beads and detection thereof by means of CSV.

a) Preparation of the First Test Solution and Hybridization

To prepare poly(A) samples for the cathodic stripping voltammetry (CSV), an aliquot of 10 μl of Dynabeads oligo(dT)₂₅ (DB) is washed twice in the same volume of binding buffer. Then 10 μl of poly(A) (depending on the monomer concentration) of known concentration are added to the binding buffer-DB mixture. The mixture is stirred for 20 min. During this, the hybridization between the poly(A) and the oligo(T) chain takes place on the surface of the DB.

b) Separation of the Hybridized Particles, Elution and Hydrolysis of the Poly(A) Chains

After the conjugation, the DB are washed several times with washing buffer. The DB are incubated in 100 μl of buffer while stirring for a further 2 min. The DB are then washed four times for 2 min each time in 100 μl of phosphate buffer (pH=7.0) and once in 100 μl of borate buffer (pH=7.7) on the stirrer. In order to elute the poly(A) chains, the DB are incubated in 10 μl of 5 mM borate buffer at 85° C. for 2 min. The poly(A) is hydrolyzed by adding 10 μl of 1 M HClO₄ and incubating at 65° C. for 30 min. The HClO₄ is subsequently neutralized by adding 20 μl of a 0.5 M NaOH solution.

c) Alternative Hybridization Method

It is also possible to apply a triple hybridization. For this purpose, the hybridization of DB and poly(A) is followed by washing the bead-poly(A) mixture twice in 10 μl of binding buffer. Poly(A) is added in the same concentration as poly(T) to the DB in order to form a duplex with the bound poly(A), and this mixture is incubated on a stirrer for 20 min. Subsequently, poly(A) is added in the same concentration as poly(A) and poly(T) in order to bind the free single-stranded ends of poly(T). The DB-containing solution is washed several times as described above. The amount of poly(A) is found by CSV measurement of adenine.

d) Electrochemical Detection of the Adenine Bases

The subsequent cathodic stripping voltammetry of adenine takes place in differential pulse mode (DPCSV). A 0.05 M borate buffer is used as electrolyte. The voltammogram of adenine in low concentration is shown in FIG. 1. The curves were obtained with a deposition potential of −0.2 V and a waiting time of 5 min. The adenine peaks are located near 0 V and migrate as the adenine concentration increases into the negative measurement range. A higher symmetry was achieved by baseline correction by the moving average method (FIG. 1b).

### 2nd Exemplary Embodiment

### Liberation of Purine Bases from DNA (or RNA) by Acid Treatment

The second exemplary embodiment shows one possibility of detecting DNA by hybridization on particles, depurination and CSV. It is additionally shown that the DNA remaining after depurination has no effect on the detection and quantification of the purine bases.

a) Release of the Purine Bases, Generation of Apurinic Acid

In this method, the DNA bound to DB is detected by CSV. The purine bases (adenine and guanine) are released by acid treatment of the DNA. Removal of the purine bases from the DNA structure results in breaks in the DNA strands, generating apurinic acid (APA) and free adenine and guanine. The average molecular weight of APA is about 15 000. Adenine and guanine can be detected in nanomolar concentrations by CSV.

The advantage of this method is the extremely high sensitivity in the determination of DNA.

APA is obtained by 24-hour dialysis of calf thymus DNA against 0.1 M HCl. Adenine and guanine are eliminated from the DNA sugar-phosphate backbone and separated from APA by dialysis. Complete removal of the bases was checked by voltammetry.

B) Electrochemical Detection by CSV, Effect of APA

The effect of APA on the adenine detection is tested by means of CSV by adding APA to the adenine solution. APA in concentrations of 1.2×10⁻⁸ M to 9.1×10⁻⁴ M is added to a 1.2×10⁻⁸ M adenine solution. Only with a 10-fold excess of APA is a slight decline in the adenine peak to be observed (FIG. 2). With the highest excess of APA of 10⁴ the adenine peak increased slightly by about 12%, which is probably attributable to traces of uneliminated purine bases in APA.

The results show that DNA can be detected in very low concentrations after removal of the purine bases by means of CSV. The detection is based on the CSV peaks of the purine bases in the presence of APA. The APA has no effect on the DNA detection

Calf thymus DNA contains about 57% adenine and 43% guanine. A depurination would therefore necessarily result in the same base ratio. Comparison of the DPCSV signals obtained after the depurination of the DNA with the signal at an identical concentration of adenine and guanine shows that the peak in the case of the adenine-guanine mixture is only 10% higher than with depurinated DNA in equimolar concentrations (FIG. 3a). The depurination of single- and double-stranded DNA produces as expected no difference in the DPSCV [sic] (FIG. 3b).

### Exemplary Embodiment 3

### Electrochemical Detection of Specific Hybridization of Nucleic Acids on Magnetic Beads

The third exemplary embodiment shows the specificity of the hybridization on particle surfaces.

In order to check the specificity of the hybridization, 25 bases of poly(T) ODN are bound to the DB and hybridized with poly(A) or nonspecific calf thymus (CT) DNA. For this purpose, 15 μl of 3.1×10⁻⁴ M poly(A) and CT DNA is hybridized in 15 μl of BD binding buffer. After the hybridization, the DB are extracted from the solution and analyzed by adenine CSV in analogy to exemplary embodiment 1.

Analysis of the height of the adenine peaks proves the specificity of the hybridization. The hybridization with poly(A) shows a clear adenine peak, and only a small background signal can be measured after hybridization with CT DNA, although the CT DNA has a high adenine content (FIG. 4).

1. A method for detecting and/or quantifying an analyte in a liquid, in particular nucleic acids, having the following steps:
a) provision of first microparticles and of a probe which has a specific affinity for the analyte and for the first microparticles, or of second microparticles having the probe linked to the surface thereof,
b) preparation of a first solution containing the analyte, the probe and the first microparticles under conditions under which the probe binds to the analyte and to the first microparticles, or of a first solution containing the analyte and the second microparticles under conditions under which the analyte binds to the probe,
c) separation of the first or second microparticles from the first solution,
d) detection of the analyte by means of an electrochemical method, where the first or second microparticles are transferred into a second solution for detecting the analyte.

2. The method as claimed in claim 1, where the first or second microparticles are designed to be magnetic.

3. The method as claimed in claim 1 or 2, where the probe binds to the first microparticles by means of biotin, streptavidin or avidin.

4. The method as claimed in any of the preceding claims, where the analyte or the probe is labeled with complex compounds containing osmium, preferably osmium tetroxide.

5. The method as claimed in any of the preceding claims, where the complex compound is linked, preferably terminally, to the analyte or the probe.

6. The method as claimed in any of the preceding claims, where the probe is labeled with cysteine.

7. The method as claimed in any of the preceding claims, where the binding of the analyte to the probe is followed by addition of a reporter probe labeled with cysteine or osmium complex compounds, so that the reporter probe hybridizes with a single-stranded overhang of the analyte.

8. The method as claimed in claim 7, where the reporter probe is removed from the analyte and subsequently detected electrochemically.

9. The method as claimed in any of the preceding claims, where a first antibody specific for the analyte is added to the second solution for the detection.

10. The method as claimed in any of the preceding claims, where an enzyme which chemically modifies the analyte or the first antibody, preferably peroxidase, is added to the second solution.

11. The method as claimed in the preceding claims, where a second antibody specifically binding to the first antibody is added to the second solution.

12. The method as claimed in any of the preceding claims, where the analyte in the second solution is hydrolyzed by acid.

13. The method as claimed in any of the preceding claims, where, when DNA is used as analyte, the purine bases are released in the hydrolysis.

14. The method as claimed in any of the preceding claims, where a magnetic or electric field is applied in step d) so that the first or second microparticles or the analyte are moved into the vicinity of an electrode.

15. The method as claimed in any of the preceding claims, where the first or second microparticles are bound to the electrode or kept in the vicinity thereof.

16. The method as claimed in any of the preceding claims, where an opposite magnetic or electric field is applied for a preset period so that molecules interfering with the electrochemical detection or first or second microparticles are moved away from the electrode.

17. The method as claimed in any of the preceding claims, where the application of the field and of the opposite field takes place cyclically.

18. The method as claimed in any of the preceding claims, where the electrode comprises at least one of the following materials: electrically conductive plastic, polymers, mercury, gold, carbon, indium-tin oxide.

19. The method as claimed in any of the preceding claims, where a layer or a membrane for retaining molecules of a preset size is provided on or in front of the surface and/or in front of a measurement cell containing the electrode.

20. The method as claimed in any of the preceding claims, where cathodic stripping voltammetry (CSV) is used as electrochemical detection method.

21. The method as claimed in any of the preceding claims, wherein the analyte and/or its hydrolysis products are identified by means of their specific redox characteristics by means of the electrochemical detection method.

22. The method as claimed in any of the preceding claims, where the analyte is concentrated or purified by means of a competitive assay.

23. The method as claimed in any of the preceding claims, where the analyte is amplified before or during step b) by means of a nucleic acid amplification reaction, especially a PCR.

24. The method as claimed in claim 23, where the probe is a primer employed in the amplification reaction.
